(19) United States
(12) Patent Application Publication
(10) Pub. No.: US 2024/0120052 A1
(43) Pub. Date: Apr. 11, 2024

(54) WELLBEING-IMPROVING-CLOUD (WIC)

(71) Applicant: Avi Harel, Hod-HaSaron (IL)

(72) Inventor: Avi Harel, Hod-HaSaron (IL)

(21) Appl. No.: 18/242,852

(22) Filed: Sep. 6, 2023

Related U.S. Application Data
(60) Provisional application No. 63/414,878, filed on Oct. 10, 2022.

(51) Int. Cl. G16H 20/00; G16H 10/20; G16H 40/67; G16H 50/30

(52) U.S. Cl. CPC G16H 20/00; G16H 10/20; G16H 40/67; G16H 50/30

(57) ABSTRACT

An example of a wellbeing-improving cloud (WIC) is disclosed. The WIC may comprise a wellbeing server (WS) and a Protocol-DB (PDB). An example of WS is configured to generate one or more protocols for wellbeing treatment and to store the generated protocols in the PDB. Each protocol may define a sequence of actions wherein each action comprises activating a certain sense-generator (SenG) in a certain setting for a certain period of time. In addition, the action may comprise reading the output of one or more sensors that are associated with a user's-wellbeing-improving-system (UWIS). Based on those reading the WS may modify the protocol.

### CROSS REFERENCE TO RELATED PATENT APPLICATIONS

This utility patent application being filed in the United States as a non-provisional application for patent under Title 35 U.S.C. § 100 et seq. and 37 C.F.R. § 1.53(b) and, claiming the benefit of the prior filing date under Title 35, U.S.C. § 119(e) of the United States provisional application for patent that was filed on Oct. 10, 2022 and assigned the Ser. No. 63/414,878, which application is herein incorporated by reference in its entirety. Further, this application is related to the following concurrently filed United States non-provisional application for patent, which is incorporated herein in its entirety: the application bearing the title of SYSTEM AND METHOD FOR IMPROVING THE WELLBEING OF AN INDIVIDUAL, identified by attorney docket number 03169.1011gss (AH-01-CON).

### FIELD OF THE DISCLOSURE

The present disclosure relates to the field of improving the quality of life and the wellbeing of a person in the middle age or above.

### BACKGROUND

After age of 40 a common person may start losing some of his memories. The amount of missing memories is increased along the years. Some of the missing memories are associated with the human senses. Senses such as but not limited to smelling, seeing, hearing, tasting, etc. Such a person may lose pictures from his childhood, sights that he visited in the past. Voices that were associated with his past, family members, friends, events, etc. In addition, a common person above the age 40 may suffer from depression, reduction in the ability to learn, sleeping problem, etc.

Chronic illness, such as but not limited to Alzheimer's disease, may start few years before the appearing of their symptoms. There are cases in which a chronic illness may start 10 to 20 years before the symptoms. Thus, when the symptoms appear it is already too late to treat the illness.

That reduction in the performance of the person may reduce the quality of life of that person. Therefore there is a need for a new system and method which will improve the wellbeing of such a person.

### BRIEF SUMMARY

The needs and the deficiencies that are described above are not intended to limit the scope of the inventive concepts of the present disclosure in any manner. The needs are presented for illustration only. The disclosure is directed to a novel system and method for exercising the brain of an individual in order to improve the individual wellbeing. The wellbeing treatment can be executed by manipulating one or more sense of the individual.

In an example embodiment of the disclosed technique manipulating the one or more senses can be done by using sense generators. The sense generators can be controlled by a processing unit. The processing unit can be a smart-phone, a laptop, a personal computer, etc. The sense generators may comprise tone (sound) generator, smell generator, light generator, picture generator, video generator, speech generator, self-hypnosis generator, relaxation generator, etc. An example of a tone generator can be Online-Tone-Generator by Tomasz Szynalski or Frequency-Sound-Generator by Google, etc. An example of smell generator can be “Ninu-smart-perfume” Another example of smell generator can be SmX1 manufactured by Sensory Co California, etc. A slide generator or video generator can be associated with the display of the processing device and may deliver pictures. The pictures can be delivered from the private storage of the user or from a public database (DB).

In order to measure the responses of the individual to stimulations, which are generated by those sense generators, the individual can be associated with one or more sensors. The sensors may comprise bio sensors and/or neuro sensors. The bio sensors may comprise sensors that can measure blood pressure, pulse oximeter, heart pulse rate. Additional bio-sensors may comprise smart watches, which include a plurality of sensors, sensors such as but not limited for measuring the connectivity of the skin, heart pulse rate, etc. Some of the sensors may be used for bio-feedback, etc. The neuro sensors may comprise sensors that can measure brain waves, sleeping quality, body tension, etc. Those sensors are well known to one of ordinary skill in the art and will not be further disclosed.

Following are few examples of sensors that can be used by an example embodiment of the disclosed system. A “Vibeat WearO2 Pulse Oximeter Bluetooth” or “Pulse X Pro” can be used for measuring Blood Oxygen and Heart Rate. In addition, headbands for measuring electrical signals produced by the brain can be used too. The headband can be associated with two or more electrodes that are configured to receive brain waves.

Examples of wellbeing treatment of an individual may comprise three phases. The first phase can be referred as the calibration phase or learning phase of an individual. During the first phase the system learns how the individual responds to different stimulations that were delivered simultaneously or one after the other by one or more sense generators. The individual is requested to write in a table the level of his wellbeing that is associated with each stimulation or a combination of stimulations. In other columns of the table the system may store the setting of the different sense generator and the reading of each sensor. The table can be stored on a computer readable non-transitory storage medium.

The level of the wellbeing can be rank between zero to five. Zero can indicate no wellbeing while five can indicate high wellbeing. Next, one of the parameters can be changed and the process starts again. The parameter can be the volume of the tones that are generated, the frequency range of the tones, and the duration of each cycle. In a similar way a smell generator may start with certain type of smells, for example smell of flowers, then switch to other type of smells, for example smells of flowers, smell of perfume, smell of cooking, etc. The power level of each stimulation can be changed too, etc. Thus, at the end of the learning phase the system may have a plurality of tables indicating the response of an individual-wellbeing to a plurality of stimulations.

The second phase is the processing phase. During the processing phase the processing device process the relation between the level of the stimulation, the reading of the one or more sensors and the score that was written by the individual in the table. At the end of the processing phase the processing device may generate one or more protocols for improving the wellbeing. Such a protocol may comprise the order of activating one or more sense generators, a sequence of power level in which a sense generator can be activated, the duration of each step per each generator, etc. These protocols can be stored in a DB that is associated with that individual. In some example embodiments this DB can be accessed only by that individual. In other example embodiments of the disclosed technique the plurality of private DBs can be accessed by a central server.

The 3^{rd} phase is the testing and adjusting phase. During this phase the individual can use the system while activating one of the protocols while the processing device processes the reading of the sensors and determines whether the wellbeing of the individual is improved. Than the processing unit may change the setting of one or more of the sense generators in order to check its influence on the individual and accordingly the protocol is adjusted. At the end of this phase the system is ready to be used by that individual.

In those embodiments the central server can be configured to prepare a plurality of group-protocols. The groups can be arranged based on gender, ages, education, profession, etc. Those protocols can be used as a preliminary protocol for a new user. Thus, a new user may start the learning phase with a protocol that is popular among people that belong to the same group.

In some example embodiment a certain individual may be associated with two or more protocols. Each protocol can be related to the individual current mode. For example, one protocol can be activated after physical activity. Other protocol can be activated after eating dinner, etc.

An example of a protocol may comprise a sequence of activating one or more of the sense generators, in parallel to the operation the one or more generators the processing unit can be configured to collect readings from the one or more sensors that are associated with that individual. In some embodiments the processing unit can be configured to process the reading of the sensors and determine whether the individual is in the direction to improve his wellbeing. If not, the processing unit may amend the protocol in order to improve the wellbeing of the user. In some embodiments the processing unit is configured to transfer the collected reading of the sensors toward the central server and the central server is configured to amend the protocol according to the reading from that individual.

An example of a treatment for improving the wellbeing of a user may comprise fetching an appropriate protocol for that user. Per each line of the protocol activate the one or more SenGs that are related to that line and set their controls according to the setting that is written in the protocol. Then the setting of each SenG can be change in plus/minus few levels while analyzing the readings from the one or more types of sensors (ToS) in order to find the setting that gives optimal wellbeing base on the readings from those sensors. Those setting of the one or more SenGs may replace the old setting in the relevant protocol and may be uploaded to the central server.

The foregoing summary is not intended to summarize each potential embodiment or every aspect of the present invention, and other features and advantages of the present invention will become apparent upon reading the following detailed description of the embodiments with the accompanying drawings and appended claims.

Furthermore, although specific embodiments are described in detail to illustrate the inventive concepts to a person skilled in the art, such embodiments can be modified to various modifications and alternative forms. Accordingly, the figures and written description are not intended to limit the scope of the inventive concepts in any manner.

Other objects, features, and advantages of the present invention will become apparent upon reading the following detailed description of the embodiments with the accompanying drawings and appended claims.

### BRIEF DESCRIPTION OF THE DRAWINGS

Examples of embodiments of the present disclosure will be understood and appreciated more fully from the following detailed description, taken in conjunction with the drawings in which:

FIG. 1 illustrates a simplified block diagram with relevant elements of an example embodiment of a system for improving the wellbeing of a person that operates according to the disclosed technique;

FIG. 2 schematically illustrates a flowchart showing relevant processes that can be implemented during phase one in order to learn the individual's responds to different stimulations.

FIG. 3 schematically illustrates a flowchart showing relevant processes that can be executed by the central server on data obtained from a user-wellbeing-improving-controller (UWIC);

FIG. 4 schematically illustrates a flowchart showing relevant processes that can be implemented by the server in order to generate a group-protocol per a group of individuals; and

FIG. 5 schematically illustrates a flowchart showing relevant processes that can be implemented by a processing unit during wellbeing treatment of an individual.

### DESCRIPTION OF EMBODIMENTS

Turning now to the figures in which like numerals represent like elements throughout the several views, of embodiments of the present disclosure that are described. For convenience, only some elements of the same group may be labeled with numerals. The purpose of the drawings is to describe exemplary embodiments and is not for production purpose. Therefore features shown in the figures were chosen only for convenience and clarity of understanding.

In the following description, for purposes of explanation, numerous specific details are set forth in order to provide a thorough understanding of the invention. It will be apparent, however, to one skilled in the art that the invention may be practiced without these specific details. In other instances, structure and devices are shown in block diagram form in order to avoid obscuring the invention. Moreover, the language used in this disclosure has been principally selected for readability and instructional purposes, and may not have been selected to delineate or circumscribe the inventive subject matter, resort to the claims being necessary to determine such inventive subject matter.

Reference in the specification to “one embodiment” or to “an embodiment” means that a particular feature, structure, or characteristic described in connection with the embodiments is included in at least one embodiment of the invention, and multiple references to “one embodiment” or “an embodiment” should not be understood as necessarily all referring to the same embodiment.

In this specification, these implementations, or any other form that the invention may take, may be referred to as techniques. In general, the order of the steps of disclosed processes may be altered within the scope of the invention. Unless stated otherwise, a component such as a processor, a server, a control unit or a non-transitory computer readable storage device described as being configured to perform a task may be implemented as a general component that is temporarily configured to perform the task at a given time or a specific component that is manufactured to perform the task. As used herein, the term ‘processor’ can refer to a computer such as but not limited to Intel NUC, wherein NUC stands for Next-Unit-of-Computing or “Amazon EC2 A1 Instances” or “Amazon EC2 P3 Instances”, which are maintained by Amazon Crop USA, for example.

Although some of the following description is written in terms that relate to software or firmware, embodiments may implement the features and functionality described herein in software as desired, including any combination of cloud resources, 3^{rd} party virtual machines, application-program-interface (API), etc.

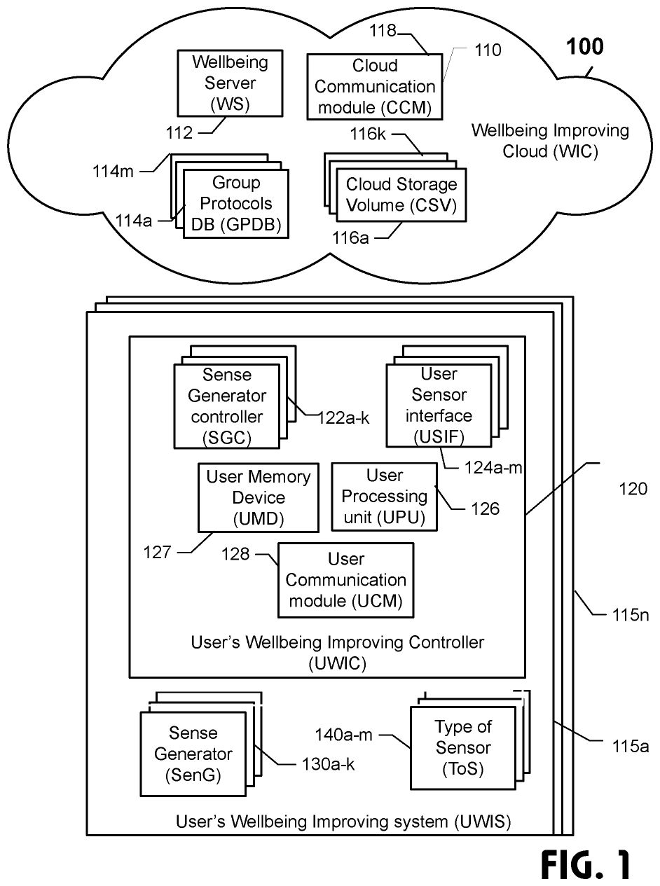

FIG. 1 depicts a block diagram with relevant elements of an example of a Wellbeing-Improving-Environment (WIE) 100 in which systems and/or methods, described herein, can be implemented. WIE 100 may comprise a Wellbeing-Improving-Cloud (WIC) 110 and one or more User's-Wellbeing-Improving-System (UWIS) 115a-n. Wherein each UWIS 115a-n may comprise a user-wellbeing-improving-controller (UWIC) 120 and one or more sense-generators (SenG) 130a-k and one or more types of sensors (ToS) 140a-m. One or more of elements of WIE 100 can comprise one or more processors that are embedded in one or more computers. The computer can be Intel NUC, wherein NUC stands for Next-Unit-of-Computing or “Amazon EC2 A1 Instances” or “Amazon EC2 P3 Instances”, which are maintained by Amazon Crop USA, for example.

An example of a WIC 110 may comprise a Wellbeing-Server (WS) 112, one or more Group-Protocol-DBs (GPDB) 114a-m, one or more cloud-storage-volume (CSV) 116a-k and a cloud-communication-module (CCM) 118. An example of WS 112 can be configured to manage the operation of the WIC 110. It can be configured to communicate, via CCM 118, with one or more sensors manufactures, sense generators manufactures, health websites, etc. in order to collect information that may be needed during the operation of the WIE 100. The collected information can be stored in CSV 116a-k.

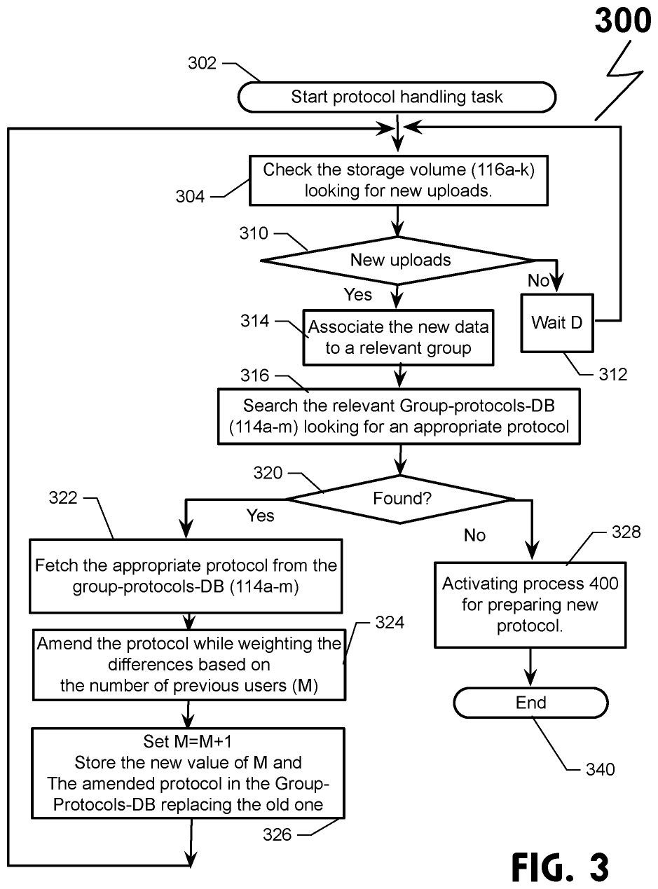

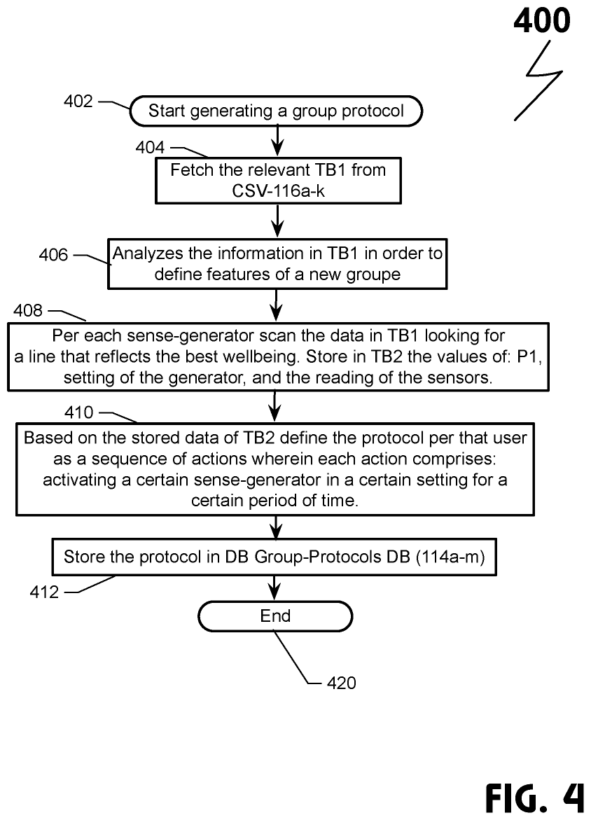

In addition some example embodiments of WS 112 can be configured to communicate, via CCM 118, with one or more UWIC 120 via user-communication module (UCM) 128 in order to download an appropriate group protocol from GPDB 114a-n, or an appropriate individual protocol from CSV 116a-k to a UMD 127 in order to start a wellbeing treatment. At the end of the wellbeing treatment the WS 112 can be configured to upload, via CCM 118, a table that was filled by the UWIC 120 and the individual during or at the end of a wellbeing treatment. More information about the operation of WS 112 is disclosed below in conjunction with FIG. 3 and FIG. 4.

The communication via CCM 118 can be implemented over a packet switch network such as but not limited to an Internet Protocol (IP) network. The IP packets can be transferred over a cellular network, a mobile network, a radio network and or over Public Switched Telephone Network (PSTN). Those networks are well known to a person having ordinary skill in the art and will not be further disclosed. GPDB 114a-m and CSV 116a-k are non-transitory computer readable storage devices comprising executable instructions that when executed cause a processor to execute a process as well as storing data that is related to certain individual or a process.

An example of a UWIC 120 may comprise one or more Sense-generator-Controllers (SGC) 122a-k, one or more user-sensor-interface (USIF) 124a-m, a user's-memory-device (UMD) 127, a user-processing-unit (UPU) 126 and UCM 128. The communication via UCM 128 can be implemented over a packet switch network such as but not limited to an Internet Protocol (IP) network. The IP packets can be transferred over a cellular network, a mobile network, a radio network and or over Public Switched Telephone Network (PSTN). By using the UCM 128 the UPU 126 can communicate with WS 112 via CCM 118. In addition, the one or more SGC 122a-k are configured to communicate with an appropriate SenG 130a-k via UCM 128, as well as one or more of the USIF are configured to communicate via UCM 128 with the appropriate ToS 140a-m.

An example of SGC 122a-k can be an application program interface (API) that is configured to control a sense generator according to a protocol that was downloaded to UMD 127. The SGC 122a-k can communicate with a manufacture of a sense generator in order to down load information about the operation of that SenG 130a-k, APIs to control that generator SenG 130a-k and APIs to monitor its operation. The control information for a smell generator 130a-k may comprise the type of smells: smell of flowers, smells of a street, smell of perfume, etc. The control information may further comprise the power and the duration of each stimulation, etc. The control information for a sound generator 130a-k may comprise the frequency of each tone, the volume, the duration, etc.

SenG 130a-k may comprise generators such as but not limited to: smell generators, light generators, picture generator, video generator, speech generator, tone (sound) generator, self-hypnosis generator, relaxation generator, etc. An example of a tone generator can be Online-Tone-Generator by Tomasz Szynalski or Frequency-Sound-Generator by Google, etc. An example of smell generator 130a-k can be “Ninu-smart-perfume” Another example of smell generator 130a-k can be SmX1 manufactured by SensoryCo California, etc. A slide generator or video generator 130a-k can be associated with the display of the UWIC 120 and may deliver pictures. The pictures can be delivered from the UMD 127 or from CSV 116a-k or from a public database.

An example of USIF 124a-m can be an application program interface (API) that is configured to read the measuring of the one or more ToS 140a-m according to a protocol that was downloaded to UMD 127. The USIF 124a-m can communicate with a manufacture of a sensor in order to down load information about the operation of that sensor, APIs to control that sensor 140a-m and APIs to read information from that sensor ToS 140a-m.

ToS 140a-m may comprise bio sensors and/or neuro sensors. The bio sensors may comprise sensors that can measure blood pressure, pulse oximeter, heart pulse rate, smart watches that includes a plurality of sensors, connectivity of the skin, bio-feedback sensors, etc. The neuro sensors may comprise sensors that can measure brain waves, sleeping quality, body tension, etc. Those sensors are well known to one of ordinary skill in the art and will not be further disclosed.

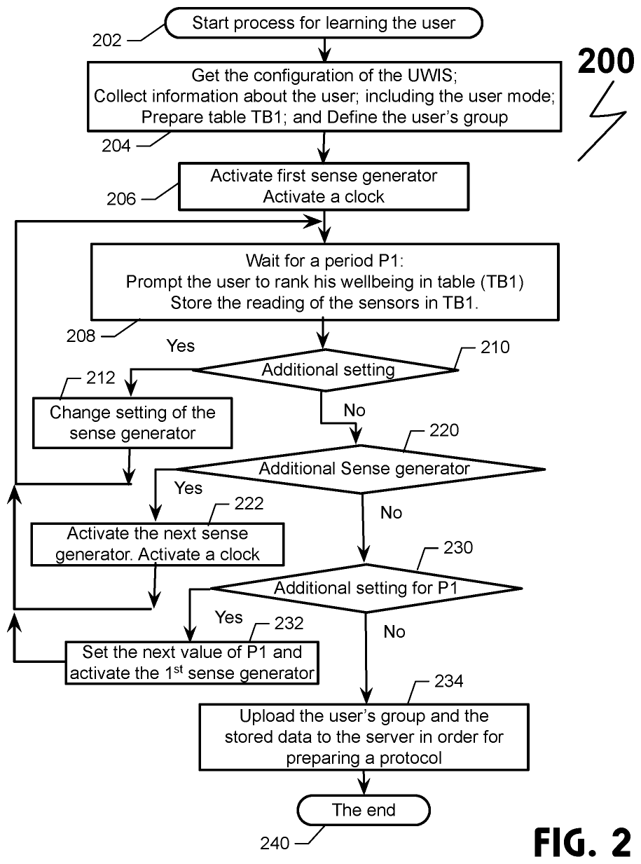

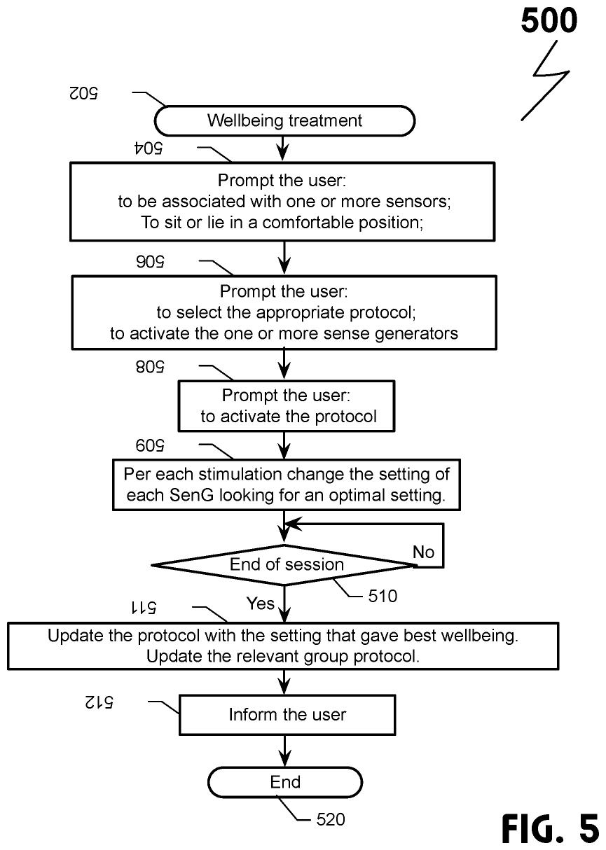

An example of UPU 126 can comprise one or more processors that are embedded in a computer. The computer can be a cellular phone, a hand held computer, a Personal-digital-assistant (PDA), a laptop, etc. An example of UPU 126 can be configured to control the operation the relevant elements of UWIC 120. UPU 126 may communicate with WS 112 in order to download one or more protocols from GPDB 114a-m in order to execute a wellbeing treatment. According to the downloaded protocol the UPU 126 may instruct the SGC 122a-k to activate the appropriate SenG 130a-k. In addition UPU 126 may instruct the USIF 124a-m to read the reading of the appropriate ToS 140a-m. More information about the operation of UPU 126 is disclosed below in conjunction with FIG. 2 and FIG. 5.

FIG. 2 is a flowchart showing relevant processes that can be executed by method 200. Method 200 can be implemented by an example UPU 126 during phase one in order to learn how a new user responds to different stimulations and the configuration of the UWIS 115a-n. Method 200 can be initiated 202 by the user. After initiation at block 204 the user can be prompted to enter information related to the user. Information such as age, gender, profession, information about the user's physical condition, current mode of the user, etc. The mode of the user can be such as but not limited to after-work, after-exercising, during-exercising, after-dinner, etc. After loading the user's related information, the user can be prompted 204 to load information regarding the configuration of the relevant UWIS 115a-n. The information may comprise information about UWIC 120 and the relevant SenG 130a-k and ToS 140a-m. At the end of process 204 a table TB1 can be prepared.

TB1 may have a plurality of lines and columns. Each line can be associated with one period of the learning process 200. The first column can be associated with the duration of that period in minutes, the following columns can be associated with the setting of each SenG 130a-k, the next columns can be associated to the reading of the ToS 140a-m and the last column can be assigned to user wellbeing. In UWIS 115a-n in which the ULU 126 can control the one or more SenG 130a-k and be able to read the one or more ToS 140a-m. In those UWIS 115a-n the ULU 126 can be configured to fill the columns of TB1 that are related to the setting of the SenG 130a-k and the reading of the ToS 140a-m. In other UWIS 115a-n the user is requested to fill those columns.

Next, method 200 may start learning how the user responds to different setting of different SenG 130a-k. At block 206 a first SenG can be set to a first setting. Then, the SenG and a clock can be activated simultaneously. At the end of period P1 process 200 may present TB1 on a display of the UPU 126 and prompt 208 the user to rank the level of the user's wellbeing in the appropriate cell of TB1. The level of the wellbeing can be rank between zero to five. Zero can indicate no wellbeing while five can indicate high wellbeing. In parallel reading of the ToS 140 a-m can be stored in the appropriate cells of TB1. P1 can be in the range of few minutes, 5-10 minutes, for example.

In some example embodiments of process 200 the level of the wellbeing can be defined by the UPU 126 based on the readings from the one or more ToSs 140a-m that are currently associated with the user. The determined level of the wellbeing can be done by comparing the current readings of the ToSs 140a-m to data stored in the UMD 127 or in the CSV 116a-k. The stored data can be produced in the past by the current user or by other users that belong to the same group as the current user.

At block 210 a decision is made whether the current SenG 140a-k has additional setting. If yes 210 then process 200 may change 212 the setting and returns to block 208. If 210 the decision is no more setting, then at block 220 a decision is made whether there are additional SenGs. If 220 yes, then ULU 126 may activate 222 the next SenG 130a-k and the clock and process 200 returns to block 208.

If 220 there are no additional SenG 130a-k, then at block 230 a decision is made whether there are additional setting per P1. In such example embodiment of process 200 the value of P1 may have few setting, 5 minutes, 10 minutes, 15 minutes, and 20 minutes, for example. In such embodiment at block 230 a decision is made whether P1 has additional possible setting. If 230 yes, then at block 232 the value of P1 is changed to the next possible setting and the first SenG 130a-k can be activated and process 200 may return to block 208.

If 230 there are no additional setting per period P1, then then at block 234 the information about the user, the configuration of the UWIS 115a-n and TB1 can be uploaded 234 to WS 112 with a request to prepare one or more protocols for that user. Then method 200 can be terminated 240.

Some example embodiments of process 200 can be modified in order to learn how the user responds to stimulations that are generated by two or more SenGs 130a-k. In such embodiment block 206 can be configured to activate two SenGs 130a-k simultaneously. An image generator can be activating with smell generator in order to present images of flowers with their smell, for example. Block 210 and 212 can be modified to change the setting of the two SneGs 130a-k. Changing the setting of the two SenG can be done one after the other, etc.

FIG. 3 illustrates flowchart showing relevant processes that can be executed by process 300 in order to handle a protocol. Process 300 can be implemented by WS 112 upon receiving a copy of TB1 from a UWIC 120. Method 300 can be initiated 302 by WS 112 and may run in a loop. After initiation 302 the CSV 116a-k (FIG. 1) can be searched 304 looking for a new upload. If 310 there is no new upload then process may wait 312 for D seconds, for example. The value of D can be in the range of zero to 120 seconds, for example. At the end of the waiting period process 300 may return to block 304.

If 310 a new upload of TB1 was found, then the obtained new data is processed 314 in order to associate the new data to an appropriate group. The decision can be based on the user that generates the obtained TB1. It can be based on one or more features of the user. Features such as but not limited to, gender, profession, information about the user's physical condition, current mode of the user, etc. Upon determining which group can be associated to that user an appropriate protocol can be fetched 322 from GPDB 114a-m.

Next, at block 324 the WS 112 may amend the fetched protocol based on the data stored in the uploaded TB1. An example embodiment of WS 112 may calculate a new value 324 of a parameter in the protocol as the weighted average between the old value, while considering the amount of previous users that were involve in amending the relevant protocol, and the new value. The calculated new value can be stored in the protocol and the protocol can be stored in GPDB 114a-m, the value of M can be incremented by one and be stored 326 in association with the amended protocol. The value of M represents the number of users that uploaded their TB1 before the current user. Then process 300 may return to block 304 looking for a new upload.

Returning now to block 320, If 320 a relevant group protocol was not found, then process 400 for preparing a new protocol can be activated 328, a pointer to TB1 can be transferred to process 400 and process 300 can be terminated 340.

FIG. 4 illustrates flowchart showing relevant processes that can be executed by an example of method 400. Method 400 can be implemented by WS 112 for generating a new group protocol. Method 400 can be initiated 402 by WS 112 upon receiving TB1 from a user that cannot be assigned to any of the existing groups, see block 328 FIG. 3. Thus, a new group has to be defining by the WS 112. After initiation process 400 may fetch 404 the new uploaded TB1 from CSV-116a-k (FIG. 1). The information from TB1 can be analyzed 406 in order to obtain one or more parameters that can be used for defining a new group that can be related to that user. The information may comprise age, gender, profession, information about the user's physical condition, current mode of the user, etc.

Next at block 408, per each sense-generator 130a-k, the section of TB1 that is associated with that sense generator can be scanned looking for a setting (a line of TB1) that is associated with the best score of wellbeing of the user. The score can be given by the user or the score can be given automatically by UPU 126 after analyzing the reading of the one or more ToS 140a-m (FIG. 1). This line can be copied to a new table TB2. Thus TB2 comprises settings that got the best wellbeing score related to that user.

At block 410 a new protocol can be defined, based on TB2, as a sequence of actions wherein each section comprises activating a certain sense generator in a certain setting for a certain period of time. The new protocol can be stored 412 in GPDB 114a-m. Wherein the group can be defined based on the parameters that were defined in block 406. Then process 400 can be terminated 420.

In some example embodiments of the disclosed technique process 410 can be modified to activate two or more sense-generators in parallel. A smell generator can be activated in parallel with a slide generator in order to deliver an image of a flower with the smell of that flower, for example. An image generator can be activated with sound generator in order to deliver an image of a beach with the noise of the wave, etc. Such a combination may generate higher wellbeing at a user. In such embodiment process 408 can be modified to be executed per each combination of the SenG 130a-k. In a similar way process 410 can be modified to activating a certain combination of SenGs 130a-k, etc.

FIG. 5 schematically illustrates a flowchart showing relevant processes 500 that can be implemented by an embodiment of a user processing unit (UPU) 126 during wellbeing treatment of an individual. Process 500 can be activated 502 by a user who wishes to start a wellbeing treatment. After initiation 502 process 500 may prompt 504 the user to be associated with one or more sensors. The sensors can be bio-sensors and or neuro-sensors. Then the user can be prompted to sit or lie in a comfortable position.

Next, the user can be prompted 506 to download an appropriate protocol from GPDB 114a-m. A protocol that fits the user's group, current mode, etc. Some example embodiments of process 500 may be configured to use only one protocol. Then, the protocol can be activated 508. During the activation of the protocol the UPU 126 may instruct the one or more sense-generator to operate according to the protocol. At block 509 the setting of each SenG may be changed a little around the setting, which is written in the protocol. The small changes 509 are used in order to find an optimal setting for the current user at the current timing. Such a process may return per each SenG that is written in the protocol. At the end of the session 510, after activating the last SenG that is mentioned in the protocol, process 500 may proceed to block 511. At block 511, the updated protocol can be uploaded 511 to the WS 112 in order to amend the group protocol and the user protocol. Amending the protocol can be weighted by the number of users that belong to that group. Then, the user can be informed 512, by friendly-waking-sound, that the wellbeing treatment is terminated 520.

The optimal setting can be defined based on a feedback that is obtained from the user. The feedback can be obtained manually by the user that is prompt to indicate when the wellbeing is improving indicating that the treatment is in the right direction. If the wellbeing is not improved, then UPU 126 may modify the treatment. In some embodiments process 500 may step back to setting with a better score and may start change the setting of another SenG 130a-k.

In some embodiments the feedback can be obtained by reading the one or more ToS 140a-m. The reading of the one or more ToSs 140a-m can be analyzed by UPU 126. In such embodiment UPU 126 can be configured to compare the readings of the one or more ToSs to data stored in the UMD 127 or in the CSV 116a-k. The stored data can be produced in the past by the current user or by other users that belong to the same group as the current user. If the readings indicate that the wellbeing is improving, then the treatment proceeds in the same direction. If the wellbeing is not improved, then UPU 126 may modify the treatment. Such example embodiments of the disclosed technique are configured to adapt the wellbeing treatment according to its impact on the user. Consequently the feedback from the ToS 140a-m improves the treatment.

In some example embodiments of process 500, block 508 may comprise fine tuning process. During the fine tuning process 508 one or more setting of the relevant SenG 130a-k can be slightly modified while the UPU 126 obtains readings from the one or more ToS 120a-m. Based on those reading the UPU may determine 508 a better relaxation state of that user. The new setting can replace the old setting of that protocol.

In this disclosure the words “module,” “device,” “component,” “element” and “unit” may be used interchangeably. Anything designated module may be a stand-alone module or a specialized module. A module may be modular or have modular aspects allowing it to be easily removed and replaced with another similar module.

In the description and claims of the present disclosure, “comprise,” “include,” “have,” and conjugates thereof are used to indicate that the object or objects of the verb are not necessarily a complete listing of members, components, elements, or parts of the subject or subjects of the verb.

It will be appreciated that the above-described apparatus, systems and methods may be varied in many ways, including, changing the order of steps, and the exact implementation used. The described embodiments include different features, not all of which are required in all embodiments of the present disclosure. Moreover, some embodiments of the present disclosure use only some of the features or possible combinations of the features. Different combinations of features noted in the described embodiments will occur to a person skilled in the art. Furthermore, some embodiments of the present disclosure may be implemented by combination of features and elements that have been described in association to different embodiments along the discloser.

It is to be understood that the above description is intended to be illustrative, and not restrictive. For example, the above-described embodiments may be used in combination with each other. Many other embodiments will be apparent to those of skill in the art upon reviewing the above description.

The scope of the invention therefore should be determined with reference to the appended claims, along with the full scope of equivalents to which such claims are entitled. In the appended claims, the terms “including” and “in which” are used as the plain-English equivalents of the respective terms “comprising” and “wherein.”

1. A wellbeing-improving cloud (WIC) comprising:
a wellbeing server (WS); and
a Protocol-DB (PDB).

2. The WIC of claim 1, wherein the WS is configured to generate one or more protocols to be used for a wellbeing treatment of one or more users.

3. The WIC of claim 2, wherein a protocol from the one or more protocols comprises a sequence of actions wherein each action comprises activating a certain sense-generator (SenG) in a certain setting for a certain period of time.

4. The WIC of claim 2, wherein the WS is configured to generate a protocol for wellbeing treatment by processing a table (TB1) that was obtained from a user's-wellbeing-improving-system (UWIS).

5. The WIC of claim 4, wherein TB1 comprises a plurality of lines and a plurality of columns, each line is associated with a period in which the UWIS learns the response of its associated user to a setup of one or more SenGs that are associated with the UWIS.

6. The WIC of claim 5, wherein the first column of TB1 is assigned to indicate the duration of the period.

7. The WIC of claim 5, wherein TB1 further comprising one or more columns wherein each of the one or more columns is assigned to indicate the setting of a SenG from one or more SenGs that are associated with the UWIS.

8. The WIC of claim 5, wherein TB1 further comprising additional one or more columns and each one of the additional one or more columns is assigned to present the reading from a type of sensor (ToS) from one or more ToSs that are associated with the UWIS.

9. The WIC of claim 5, wherein TB1 comprises a column that is assigned to indicate a rank of wellbeing that was obtained from the associated user during that period.

10. The WIC of claim 4, wherein per each SenG the WS is configured to scan TB1 looking for a line that reflects the best wellbeing and store in TB2 the value of that period, the setting of that SenG and the reading of the one or more ToSs.

11. The WIC of claim 4, wherein the WS is configured to generate a protocol based on the data stored in TB2.

12. A non-transitory computer readable storage device comprising executable instructions that when executed cause a processor at a wellbeing-server (WS):
i. to obtain from a user's-wellbeing-improving-system (UWIS) a table TB1;
ii. to process the data stored on TB1;
iii. to generate a protocol for a wellbeing treatment; and
iv. to store the protocol in a protocol database (PDB).

13. The non-transitory computer readable storage device of claim 12, wherein the protocol defines a sequence of actions to be executed by the UWBS.

14. The non-transitory computer readable storage device of claim 13, wherein each action comprises activating a certain sense-generator (SenG) in a certain setting for a certain period of time.

15. The non-transitory computer readable storage device of claim 14, wherein each action further comprises, at the end of the certain period, reading the indication from one or more type of sensor (ToS)s that are associated with the UWIS.

16. The non-transitory computer readable storage device of claim 12, further comprising instructions that when executed cause the processor to modify an existing protocol that is stored in the PDB.

17. The non-transitory computer readable storage device of claim 16, wherein modifying an existing protocol takes into consideration the number of users that previously used the existing protocol.

18. The non-transitory computer readable storage device of claim 12, further comprising instructions that when executed cause the processor to store the modified protocol instead of the existing protocol.
